(19) United States
(12) Patent Application Publication
(10) Pub. No.: US 2005/0048570 A1
(43) Pub. Date: Mar. 3, 2005

(54) Structured-functional bonding matrices for biomolecules

(75) Inventors: Achim Weber, Altbach (DE); Thomas Schiestel, Stuttgart (DE); Gunter Tovar, Stuttgart (DE); Herwig Brunner, Stuttgart (DE); Steffen Rupp, Stuttgart (DE); Nicole Hauser, Heidelberg (DE)

Correspondence Address: OSTROLENK FABER GERB & SOFFEN, 1180 AVENUE OF THE AMERICAS, NEW YORK, NY, 100368403, US

(73) Assignee: FRAUNHOFER-GESELLSCHAFT ZUR FORDERUNG DER ANGEWANDTEN

(21) Appl. No.: 10/500,425

(22) PCT Filed: Dec. 27, 2002
(86) PCT No.: PCT/EP02/14769

(30) Foreign Application Priority Data
Dec. 28, 2001 (DE) 101 64 309.8

(51) Int. Cl. G01N 33/53; C12M 1/34

(52) U.S. Cl. 435/7.1; 435/287.2

(57) ABSTRACT

The present invention relates to functional elements that comprise microstructures containing biofunctionalized nanoparticles arranged on a carrier, a method for the production of these functional elements and the use thereof.

The present invention relates to functional elements that comprise microstructures consisting of biofunctionalizable or biofunctionalized nanoparticles arranged on a carrier, methods for the production of these functional elements and the use thereof.

The investigation of biological molecules such as DNA or proteins is becoming increasingly important in a greater variety of areas, for example in environmental analysis for detecting microorganisms, in clinical diagnostics for identifying pathogens or for determining resistance to medicinal drugs, etc. Regardless of the particular application, these methods of analysis must always meet the same requirements. In particular they must be quick and inexpensive in execution, but at the same time they must be very sensitive and must give reliably reproducible results.

The so-called biochips represent a milestone in the analysis of biologically active molecules. For example, by using gene chips, which are also called DNA arrays, the determination of nucleic acids in test samples can be greatly simplified, accelerated and automated, it can be effected in parallel and it can be made more precise. These gene chips are miniaturized carriers, on whose surface nucleic acids of a known sequence are immobilized or synthesized in an ordered array. Gene chips are preferably used in the clinical diagnosis of infectious diseases, cancers and hereditary diseases. The efficiency of these gene chips in the analysis of samples is due in particular to the fact that only the tiniest of sample volumes are required and the evaluation can be effected using highly sensitive measurement techniques. Using these chips, therefore, parallel investigation of large numbers of samples becomes possible (high-throughput screening).

Protein arrays are also known. In these, as in gene chips, proteins or peptides are arranged in an ordered, known array on for example plastics membranes. Protein arrays of this kind are mainly used for investigating the mutual binding of proteins, for example receptor-ligand interactions, for identifying intracellular protein complexes, for investigating DNA-protein and RNA-protein interactions or for the analysis of protein-antibody interactions. Using protein-chip technology, it has already been possible to identify numerous protein markers for cancers or for diseases like Alzheimer's disease.

Biological molecules bound to a carrier also play an important role in the development of biocomputers. The “biocomputer” involves, in particular, the replacement of traditional electronic components like transistors, diodes, switches etc., by components that, at least partly, consist of or contain organic materials. The use of biological molecules as electronic components mainly pursues the objective of utilizing the capabilities of information-processing biological systems, for example pattern and object recognition, reproduction and self-organization. Traditional silicon-based components do not possess these capabilities. Furthermore, by using biological molecules it is possible to achieve a degree of miniaturization of electronic components that cannot even be approximated with silicon components. Component miniaturization involves a significant improvement of all the significant parameters of chips such as integration density, power dissipation, switching speed and costs by orders of magnitude. Admittedly the limits of miniaturization have not yet been reached with silicon-based components, but they are already foreseeable, because with increasing degree of miniaturization, in particular with increasing packing density, problems arise such as removal of heat, and undesirable electrical effects.

A large part of research in bioelectronics is now focused on the use of proteins as base structures for molecular components. Proteins display a high variability in their function and a high stability in their structure. When using proteins, the following strategies are followed: investigation and modification of the functional mechanisms of proteins for logic operations, utilization of structural properties for application as carrier molecules for different kinds of molecular elements and application of the structural and functional properties of proteins for the manufacture of molecular circuits. At present, in particular photoreceptor proteins are being investigated, which are able to convert light into a signal directly. This process includes the formation of an electric dipole and is accompanied by a color change of the protein. On the basis of these optoelectrical properties, such proteins can be used as “smart materials”. The best investigated example to date is bacteriorhodopsin, and the aim is to use it as an optical memory in optical information processing.

These bioelectronic components can also find application together with traditional electronic components in “mixed” computer systems. However, the bioelectronic components can also be used in other technical fields, for example in medical engineering. The use of biological materials in medical measuring and monitoring and for artificial organ transplants and prostheses offers many decisive advantages. For example, another aim is to develop effective visual aids for persons who have lost their sight. Thus, so-called retina chips are currently being developed, to be used as an implant in the human eye.

The structuring of layers on carrier materials is of greater importance for generating sensors and screening systems in the various areas of analysis. Several methods have been described for coating carriers made of metals, polymers, glass, semiconductors or ceramics with self-assembled monolayers, other monomolecular coatings, paints, metal films or polymer films. Thus, the deposition of non-functionalized particles for optical applications for the structuring of planar surfaces is known (Chen, Jiang, Kimerling and Hammond, Langmuir, 16 (2000), 7825-7834).

Fan et al. (Nature, 405 (2000), 56-60) describe the production of functional, hierarchically organized structures, combining the self-alignment of silica-surfactant systems with fast printing processes, in particular pen lithography, inkjet printing and dipole-coating of structured self-assembled monolayers (SAM). The process described can be used for the production of sensor arrays and fluid or photon systems. For the process described it is important to use stable, homogeneous ink mixtures, which after evaporation can form the desired, organically modified silica-surfactant mesophase in a self-assembly process. For this, an oligomeric silica sol is prepared in ethanol/water at a hydronium ion concentration that minimizes the siloxane condensation rate and thus permits surface self-alignment of the silica-surfactant system during the structuring process. With the ink liquid, a pattern is produced on a surface, and the preferential evaporation of ethanol causes enrichment of water, surfactant and silicates, so that a complex three-dimensional concentration gradient forms. When the critical micelle concentration (c.m.c.) is exceeded, micelles are produced, and as evaporation continues, especially of water, the continual self-organization of the micelles to silica-surfactant crystals is induced at the liquid-vapor interfaces. Hydrophobic, alcohol-soluble molecules can, after ethanol evaporation, be compartmentalized in the network of the resultant mesophase. Using micropen lithography (MPL), ordered nanostructures containing functions can thus be produced. The production of such structures using functionalizable nanoparticles by the micropen lithography process was not described.

However, the coating of carriers with biological molecules, especially structured coating, is much more complicated. The immobilization of biomolecules on a carrier is generally effected by adsorption or covalent bonding. On the one hand, the individual molecules must be positioned at high packing density at defined spacing. On the other hand the molecules must be stabilized and bound to the carrier in such a way that their biological activity is not altered or lost. Thus, in the immobilization of proteins, the three-dimensional structure that is necessary for biological activity, for example the three-dimensional structure at the active site of an enzyme, must not be altered.

U.S. Pat. No. 5,609,907 describes the production of macroscopic metallic surfaces using self-aligning colloidal metal particles. In this case, streptavidin-labeled Au colloids are applied unstructured on biotinylated SAM (self-assembled monolayer) surfaces. Owing to the negative charge on the colloidal particles there is mutual repulsion of the particles. In this way aggregation of several particles is prevented and the particles are arranged as individual particles a uniform distance apart. Accordingly, continuous structures comprising several particles cannot be produced using this technique.

The production of adhesive templates, especially the structured arrangement of biotin-functionalized particles on SAM (self-assembled monolayer) surfaces, is described by Harnett et al. (Harnett, Satyalakshmi and Craighead, Langmuir, 17 (2001), 178-182). In this, SAM surfaces are structured by electron-beam lithography and then biotinylated polystyrene particles (20 nm to 20 μm) are applied. In the method described, an inert monolayer is treated by electron-beam lithography, structures being produced. Then the treated and cleaned structures are backfilled with a reactive monolayer, to which linker molecules or polystyrene particles with terminal biotin units are bound selectively. The particles used are fluorescent, can be functionalized for example with proteins and are suitable in particular for cell-adhesion applications. The reactive monolayer used for backfilling the structures can then be removed again, i.e. the backfilling process can be reversed. In particular the method described takes advantage of the rapidity of electron-beam lithography for printing individual lines or individual points.

The production of adhesive templates by treating monolayers with amine groups on carbon nanotubes using electron-beam lithography followed by backfilling with a reactive monolayer is also known (Harnett, Satyalakshmi and Craighead, Appl. Phys. Lett., 76 (2000), 2466-2468). Works on non-electron-beam lithography include the treatment of monolayers by scanned-probe lithography (Sugimura and Nakagiri, J. Vac. Sci. Technol., 15 (B 1997), 1394-1397; Wadu-Mesthrige et al., Langmuir, 15 (1999), 8580-858), the backfilling of photolithographically treated hydrophobic monolayers with amines to produce DNA arrays (Brockman, Frutos and Corn, J. Am. Chem. Soc., 121 (1999), 8044-8051) and routine backfilling for functionalization of unprinted areas in the microcontact printing process.

Whereas the immobilization of nucleic acids like DNA on carriers can be relatively problem-free, the situation is much more complicated in the case of proteins. In particular, at present there are only a few methods by which directed immobilization of a protein, i.e. especially with preservation of. its functionality, is possible. In most of the known methods proteins are bound to a surface primarily by unspecific interactions, i.e. directed covalent bonding does not take place.

The coating of surfaces with hydrogels, which can have various functionalities, for example protein functions, is known. Coating with hydrogels does indeed lead to an increase in surface area in comparison with planar surfaces, but the unspecific fixation of the functionalities is intensified at the same time.

The present invention is based on the technical problem of developing means and methods for the production of miniaturized carrier systems with biological molecules immobilized thereon, for example gene chips and protein chips, in which the disadvantages known in the state of the art have been eliminated and in which the biomolecules are immobilized or can be immobilized at high packing density on a carrier in particular while preserving their biological activity, and which are suitable for use in a wide variety of screening and analytical systems, for example in medical measurement and monitoring, and in biocomputers.

The present invention solves the aforementioned technical problem by providing a functional element comprising a carrier with a surface and at least one microstructure arranged on the carrier surface, the said microstructure consisting of individual components in the form of nanoparticles, which have molecule-specific recognition sites that make the microstructure addressable.

The present invention thus provides a functional element with one or more microstructures arranged on its surface, with each microstructure consisting of several nanoparticles with identical or non-identical molecule-specific recognition sites. The microstructures of the functional elements can have or can be provided with biofunctions. That is, the molecule-specific recognition sites of the nanoparticles forming the microstructure can recognize, and bind, corresponding molecules, in particular organic molecules with a biological function or activity. These molecules can be nucleic acids or proteins, for example. Other molecules, for example molecules to be analyzed in a sample, can then bind to the molecules that are bound to the molecule-specific recognition sites of the nanoparticles. In contrast to the systems known in the state of the art, for example conventional gene or protein arrays, the present invention thus envisages immobilizing biological molecules on the surface of nanoparticles, rather than binding them to a planar surface directly, the said nanoparticles being used, before or after immobilization, for forming a microstructure.

The functional elements according to the invention, comprising nanoparticle systems with molecule-specific recognition sequences for binding biological molecules, offer several decisive advantages over conventional systems, for example those in which the biological molecules are immobilized on the carrier directly.

The nanoparticles used according to the invention are extremely flexible, inert systems. For example, they can consist of a great variety of cores, e.g. organic polymers or inorganic materials. Inorganic nanoparticles such as silica particles offer the advantage that they are exceptionally inert chemically and are mechanically stable. Whereas surfmers and molecularly imprinted polymers possess soft cores, nanoparticles with silica or iron cores do not swell in solvents. Swellingproof particles do not change their morphology, even if they are suspended in solvents repeatedly for an extended period. Functional elements according to the invention, comprising swellingproof particles, can therefore be used without any problems in methods of analysis or microstructurization that require the use of solvents, without the condition of the nanoparticles or of the immobilized biological molecules being adversely affected. Functional elements that contain the said nanoparticles can therefore also be used for purifying the biological molecules that are to be immobilized from complex mixtures of substances containing unwanted substances such as detergents or salts, and the molecules that are to be immobilized can be separated from the said mixtures of substances in an optimum manner in washing processes of any desired duration. On the other hand, superparamagnetic or ferromagnetic nanoparticles with an iron oxide core can line up along the field lines in a magnetic field. This property of iron oxide nanoparticles can be utilized for building up microstructures directly, in particular nanoscopic conducting paths.

The functional elements according to the invention can be used for the immobilization of a great variety of biological molecules, while preserving their biological activity. The nanoparticles used for forming the microstructures have molecule-specific recognition sites, in particular functional chemical groups, which are able to bind the molecule that is to be immobilized in such a way that the regions of the molecule necessary for the biological activity are in a state that corresponds to the native molecular state. Depending on the functional groups present on the surface of the nanoparticles, the biomolecules can be bound to the nanoparticles covalently and/or non-covalently, as required. The nanoparticles can have various functional groups, so that either different biomolecules or biomolecules with different functional groups can be immobilized with a preferred orientation. The biomolecules can be immobilized on the nanoparticles either unoriented or oriented, with almost any desired orientation of the biomolecules being possible. Stabilization of the biomolecules is achieved by immobilizing them on the nanoparticles.

The nanoparticles used for forming the microstructures possess a comparatively very high surface/volume-ratio and can accordingly bind a large amount of a biological molecule per unit mass. In comparison with systems in which biological molecules are bound to a planar carrier directly, a functional element can thus bind a much larger quantity of the biological molecules per unit area. The quantity of molecules bound per unit area, i.e. the packing density, can be further increased by superimposing several layers of particles for production of the microstructure on the carrier surface. A further increase in the quantity of biological molecules bound per unit area can be achieved by coating the nanoparticles with hydrogels first, and then with biological molecules.

The nanoparticles used according to the invention have a diameter of 5 nm to 500 nm. Using the said nanoparticles it is therefore possible to produce functional elements that have very small microstructures of any form in the nanometer to micrometer range. Use of the nanoparticles for the production of microstructures therefore permits miniaturization of the functional elements that has not been achieved previously, with considerable improvements in significant parameters of the functional elements.

The nanoparticles used according to the invention display very good adhesion on the materials that are used for making carriers or carrier surfaces. The particles can therefore be used without any problems for a large number of carrier systems and hence for a large number of different functional elements with the most varied fields of application. The microstructures formed using the nanoparticles are very homogeneous, which leads to space-independent signal intensity.

The functional elements according to the invention can have various microstructures on their carrier surface, which consist of different nanoparticles with different molecule-specific recognition sites. Accordingly, these different microstructures can also have different biofunctions. The functional elements can thus contain microstructures next to one another that contain different biological molecules or can be provided therewith. A functional element can therefore contain, for example, several different proteins or several different nucleic acids or proteins and nucleic acids simultaneously.

The functional elements according to the invention can be produced in a simple way using known methods. For example, stable suspensions can be produced very simply from nanoparticles using suitable suspending agents. Nanoparticle suspensions behave like solutions and are therefore compatible with microstructurization techniques. Nanoparticle suspensions can therefore be deposited directly, for example using conventional methods such as needle-ring printers, lithographic methods, ink-jet methods and/or microcontact methods, structured on suitable carriers, that have been pretreated with a bonding agent for firm adhesion of the nanoparticles. With an appropriate choice of bonding agent, the microstructure formed can be designed in such a way that at a later time it can be detached partially or completely from the carrier surface of the functional element, for example by altering the pH value or the temperature, and can if necessary be transferred to the carrier surface of another functional element.

The functional elements according to the invention can be implemented in the most varied manner and can therefore be used in very varied areas. For example, the functional elements according to the invention can be biochips, for example gene or protein arrays, which are used in medical analysis or diagnostics. The functional elements according to the invention can, however, also be used as an electronic component, for example as a molecular circuit, in medical measurement and monitoring or in a biocomputer.

In connection with the present invention, “functional element” means an element which, either alone or as a component part of a more complex device, i.e. in combination with other similar or different functional elements, performs at least one defined function. A functional element comprises several components, which can consist of the same or different materials. The individual components of a functional element can perform different functions within a functional element and can contribute to the overall function of the element to a varying extent or in a different manner. In the present invention a functional element comprises a carrier with a carrier surface, on which a defined layer or layers of nanoparticles is/are arranged as microstructure(s), and the said nanoparticles are provided with, and/or can be provided with, biological functions, for example biological molecules such as nucleic acids, proteins and/or PNA molecules.

The term “carrier” means that component part of the functional element that mainly determines the volume and the external form of the functional element. The term “carrier” signifies in particular a solid matrix. The carrier can be of any size and any shape, for example a sphere, a cylinder, a bar, a wire, a plate or a film. The carrier can be both a hollow body and a solid body. “Solid body” means in particular a body that essentially has no hollow spaces and can consist entirely of one material or a material combination. The solid body can also consist of a series of layers of identical or different materials.

According to the invention, the carrier of the functional element, especially the carrier surface, consists of a metal, a metal oxide, a polymer, glass, a semiconductor material or ceramic. In connection with the invention this means that either the carrier consists entirely of one of the aforesaid materials or contains this essentially or consists entirely of a combination of these materials or contains this essentially or that the surface of the carrier consists entirely of one of the aforesaid materials or contains this essentially or consists entirely of a combination of these materials or contains this essentially. The carrier or its surface then consists to at least about 60%, preferably to about 70%, more preferably to about 80% and most preferably to about 100% of one of the aforesaid materials or a combination of the said materials.

In a preferred embodiment the carrier of the functional element consists of materials such as transparent glass, silicon dioxide, metals, metal oxides, polymers and copolymers of dextrans or amides, for example acrylamide derivatives, cellulose, nylon, or polymeric materials, such as polyethylene terephthalate, cellulose acetate, polystyrene or polymethyl methacrylate or a polycarbonate of bisphenol A.

It is envisaged, according to the invention, that the surface of the functional element carrier is planar or is even prestructured, for example contains lead-ins and lead-outs. It is also envisaged according to the invention that the areas of the carrier surface not covered by the microstructure contain functionalities or chemical compounds, which prevent nonspecific attachment of biomolecules to these areas. In particular, this can be an ethylene oxide layer.

In a preferred embodiment of the invention, it is envisaged that at least one layer of a bonding agent is arranged between the carrier surface and the microstructure. The bonding agent provides firm bonding of the nanoparticles to the carrier surface of the functional element. The choice of bonding agent depends on the surface of the carrier material and the nanoparticles that are to be bonded. The bonding agent preferably consists of charged or uncharged polymers. The polymer can also be a hydrogel. The bonding agent can also be a plasma layer with charged groups, such as a polyelectrolyte, or a plasma layer with chemically reactive groups. The bonding agent can also be a silane-based or thiol-based self-assembled monolayer. It is envisaged according to the invention that the layer of bonding agent consists of at least one layer of a bonding agent. The layer of bonding agent can also, however, consist of several layers of different bonding agents, e.g. of an anionic plasma layer and a cationic polymer layer or of several polymer layers which are alternately anionic and cationic.

Another preferred embodiment of the invention relates to bonding agents whose properties, for example their cohesive properties, can be altered by an external stimulus and are therefore switchable from outside. For example, the cohesive properties of the bonding agent can be lowered by altering the pH value, the ion concentration and/or the temperature to such an extent that the microstructures bound to the carrier surface of the functional element using the bonding agent are detached and can if required be transferred to the carrier surface of another functional element.

In a further preferred embodiment of the invention it is envisaged that the carrier, especially the carrier surface, is pretreated with a surface-activating agent before applying the bonding layers and microstructures, in order to improve the bonding of the bonding layers and microstructures that are to be applied on the carrier or on its surface. The surfaces of the carrier can, for example, be activated by chemical methods, for example using primers or an acid or a base. Surface activation can also be effected using a plasma. The surface activation can also comprise the application of a self-assembled monolayer.

“Microstructure” means structures in the region of a few micrometers or nanometers. Especially in connection with the present invention, “microstructure” means a structure that consists of at least two individual components in the form of nanoparticles with molecule-specific recognition sites and is arranged on the surface of a carrier, with a certain portion of the carrier surface being covered, which has a defined shape and a defined area and is smaller than the carrier surface. According to the invention it is envisaged in particular that at least one of the area-length parameters, which determines the portion of the area covered by the microstructure, is in the micrometer range. If, for example, the microstructure has the shape of a circle, the diameter of the circle is in the micrometer range. If the microstructure is in the form of a rectangle, the breadth of this rectangle for example is in the micrometer range. According to the invention it is envisaged in particular that the at least one area-length parameter, determining the portion of the area covered by the microstructure, is smaller than 1 mm. As the microstructure according to the invention consists of at least two nanoparticles, the lower limit of this area-length parameter is 10 nm.

The portion of the area covered by the microstructure according to the invention can be of any geometric shape, for example that of a circle, an ellipse, a square, a rectangle or a line. The microstructure can, however, also be composed of several regular and/or irregular geometric shapes. If the functional element according to the invention is for example a gene chip or a protein array, the microstructure preferably has a circle-like or ellipse-like shape. If the functional element according to the invention is an electronic component for use in a biocomputer, the microstructure can also have a circuit-like shape. It is also envisaged according to the invention that several microstructures of the same or of different shape are arranged a certain distance apart on the carrier surface of a functional element.

According to the invention it is envisaged that the microstructures are applied for example using one needle-ring printer per ring/pin by lithographic techniques, such as photolithography or micropen lithography, inkjet techniques or microcontact printing methods on the surface of the carrier of the functional element. Selection of the method used for applying the microstructure or microstructures to the surface of the functional element is based on the surface of the carrier material, the nanoparticles that are to form the microstructure, and the subsequent application of the functional element.

In connection with the present invention, a “nanoparticle” means a particulate binding matrix which has molecule-specific recognition sites comprising first functional chemical groups. The nanoparticles used according to the invention comprise a core with a surface on which the first functional groups are arranged, which are capable of binding complementary second functional groups of a biomolecule covalently or non-covalently. Through interaction between the first and second functional groups, the biomolecule is immobilized on the nanoparticle and therefore on the microstructure of the functional element and/or can be immobilized thereon. The nanoparticles used according to the invention for forming the microstructures have a size of less than 500 nm, preferably less than 150 nm.

In connection with the present invention, “addressable” means that the microstructure can be found and/or detected again after the nanoparticles have been applied to the carrier surface. If, for example, the microstructure is applied to the carrier surface using a mask or a stamp, the address of the microstructure results on the one hand from the x and y coordinates of the region of the carrier surface predetermined by the mask or the stamp, onto which the microstructure is applied. On the other hand the address of the microstructure results from the molecule-specific recognition sites on the surface of the nanoparticles, which make it possible for the microstructure to be found again or detected. If the microstructure is biofunctionalizable, i.e. comprises nanoparticles with molecule-specific recognition sites, to which no biomolecules are bound, the microstructure can be found again and/or detected because one or more biomolecules bind specifically to the molecule-specific recognition sites of the nanoparticles forming the microstructure, but not to the portions of the carrier surface that are not covered by the microstructure. If, for example, the immobilized molecule is labeled with detection markers such as fluorophors, spin labels, gold particles, radioactive markers etc., the microstructure can be detected using appropriate detection techniques. If the microstructure has been biofunctionalized, i.e. comprises nanoparticles with one or more biomolecules already bound to their molecule-specific recognition sites, “addressable” means that these biomolecules can be found and/or detected by interaction with complementary structures of other molecules or by means of metrological techniques, in which only the microstructure consisting of nanoparticles shows corresponding signals, but not the portions of the carrier surface that are not covered by the microstructure. As the method of detection it is possible to use, for example, matrix-supported laser-desorption/ionization-time-of-flight mass spectrometry (MALDI-TOF-MS), which has developed to become an important method of analysis of a great variety of substances, and especially proteins. Other methods of detection are waveguide spectroscopy, fluorescence, impedance spectroscopy, radiometric and electrical methods.

According to the invention it is envisaged that the biological molecule is bound or immobilized or can be bound or immobilized on the surface of the nanoparticles forming the microstructure while preserving its biological activity, and preferably the molecule is or will be bound with a particular orientation. Biological activity of a molecule means all functions that it performs in an organism in its natural cellular environment. If the molecule is a protein, these can be specific catalytic or enzymatic functions, functions in the immune defense system, transport and storage function, regulatory function, transcription and translation functions and the like. If the molecule is a nucleic acid, the biological function can consist for example of the encoding of a gene product or the use of the nucleic acid as a template for the synthesis of further nucleic acid molecules or as a binding motif for regulatory proteins. “Retention of biological activity” means that after immobilization on the surface of a nanoparticle, a biological molecule can perform the same or almost the same biological functions at least to a similar extent as the same molecule in the non-immobilized state in suitable invitro conditions or the same molecule in its natural cellular environment.

In connection with the present invention, the term “oriented and immobilized” or “oriented immobilization” means that a molecule will be or is bound at defined positions within the molecule to the molecule-specific recognition sequences of a nanoparticle, in such a way that, for example, the three-dimensional structure of the domain(s) required for the biological activity is not altered relative to the non-immobilized state and that this or these domain(s), for example binding recesses for cellular reactants, is/are freely accessible to these on contact with other native cellular reactants.

“Oriented and immobilized” also means that during immobilization of a molecular species, all or nearly all individual molecules, but at least more than 80%, preferably more than 85% of all molecules on the surface of the nanoparticles forming the microstructure reproducibly assume an identical or almost identical orientation.

It is envisaged according to the invention that the biological molecule immobilized or that can be immobilized on the microstructure of the functional element according to the invention is in particular a nucleic acid, a protein, a PNA molecule, a fragment thereof or a mixture thereof.

In connection with the present invention, a nucleic acid is understood as a molecule that consists of at least two nucleotides, joined by a phosphodiester bond. The nucleic acid can be both a deoxyribonucleic acid and a ribonucleic acid. The nucleic acid can be both single-stranded and double-stranded. In the context of the present invention, a nucleic acid can thus also be an oligonucleotide. The nucleic acid bound to the microstructure of the functional element according to the invention preferably has a length of at least 10 bases. A nucleic acid can be of natural or synthetic origin. The nucleic acid can be modified by the methods of genetic engineering relative to the wild-type nucleic acid and/or can contain unnatural and/or unusual nucleic acid building blocks. The nucleic acid can be bound to molecules of a different kind, for example to proteins.

In connection with the present invention, a “protein” is a molecule that comprises at least two amino acids joined together by an amide bond. In the context of the present invention, a protein can thus also be a peptide, for example an oligopeptide, a polypeptide or for example a protein domain. Such a protein can be of natural or synthetic origin. The protein can be modified relative to the wild-type protein by the methods of genetic engineering and/or can contain unnatural and/or unusual amino acids. The protein can be derivatized relative to the wild-type form, for example it can have glycosylations, it can be shortened, it can be fused with other proteins or can be bound to molecules of another type, for example to carbohydrates. According to the invention, a protein can in particular be an enzyme, a receptor, a cytokine, an antigen or an antibody.

“Antibody” denotes a polypeptide that is essentially encoded by one or more immunoglobulin genes, or fragments thereof, which bind(s) and recognize(s) an analyte (antigen) specifically. Antibodies are for example in the form of intact immunoglobulins or as a number of fragments that were produced by cleavage with various peptidases. “Antibody” also denotes modified antibodies (e.g. oligomeric, reduced, oxidized and labeled antibodies). “Antibody” also encompasses antibody fragments, which have been produced either by modification of whole antibodies or by de-novo synthesis using DNA recombination techniques. The term “antibody” covers both intact molecules and fragments thereof, such as Fab, F(ab′)₂ and Fv, which can bind the epitope determinants.

PNA (Peptide Nucleic Acid or Polyamide Nucleic Acid) molecules are molecules that are not negatively charged, and act in the same way as DNA (Nielsen et al., 1991, Science, 254, 1497-1500; Nielsen et al., 1997, Biochemistry, 36, 5072-5077; Weiler et al., 1997, Nuc. Acids Res., 25, 2792-2799). PNA sequences comprise a polyamide skeletal structure of N-(2-aminoethyl)-glycine units and do not have any glucose units or phosphate groups.

In connection with the present invention, “molecule-specific recognition sites” are regions of the nanoparticle that permit a specific interaction between the nanoparticle and biological molecules as target molecules. The interaction can be based on directed attractive interaction between one or more pairs of first functional groups of the nanoparticle and the complementary second functional groups of the target molecules, which bind the first functional groups, i.e. of the biological molecules. Individual interacting pairs of functional groups between the nanoparticle and the biological molecule are in each case fixed spatially and arranged on the nanoparticle and the biological molecule. This fixation does not have to be a rigid arrangement, but can instead be quite flexible. The attractive interaction between the functional groups of the nanoparticles and the biological molecules can be in the form of non-covalent bonds such as van der Waals bonds, hydrogen bridges, π-π bonds, electrostatic interactions or hydrophobic interactions. Reversible covalent bonds, as well as mechanisms based on complementarity of shape or form, are also conceivable. The interactions envisaged according to the invention between the molecule-specific recognition sites of the nanoparticles and the target molecule are thus based on directed interactions between the pairs of functional groups and on the mutual spatial arrangement of these groups undergoing pairing on the nanoparticle and the target molecule. This interaction leads to an affine bond of covalent or non-covalent type between the two binding partners, in such a way that the biological molecule is immobilized on the surface of the nanoparticles forming the microstructure.

In a preferred embodiment of the present invention, the first functional groups, which are a component part of the molecule-specific recognition sites on the surface of the nanoparticle or form these, are selected from the group comprising active ester, alkyl ketone group, aldehyde group, amino group, carboxy group, epoxy group, maleinimide group, hydrazine group, hydrazide group, thiol group, thioester group, oligohistidine group, Strep-tag I, Strep-tag II, desthiobiotin, biotin, chitin, chitin derivatives, chitin binding domain, metal chelate complex, streptavidin, streptactin, avidin and neutravidin.

According to the invention it is also envisaged that the molecule-specific recognition site is a larger molecule such as a protein, an antibody etc., which contains the first functional groups. The molecule-specific recognition site can also be a molecular complex, which consists of several proteins and/or antibodies and/or nucleic acids, and at least one of these molecules contains the first functional groups. A protein can comprise for example an antibody and a protein bound thereto, as molecule-specific recognition sequence. The antibody can then also comprise a streptavidin group or a biotin group. The protein bound to the antibody can be a receptor, for example an MHC protein, cytokine, a T-cell receptor such as CD-8 protein and another that can bind a ligand. A molecular complex can also comprise several proteins and/or peptides, for example a biotinylated protein, which binds a further protein and additionally a peptide in a complex.

The second functional group, i.e. the functional group of the biomolecule that is to be immobilized, is selected according to the invention from the group comprising active ester, alkyl ketone group, aldehyde group, amino group, carboxy group, epoxy group, maleinimide group, hydrazine group, hydrazide group, thiol group, thioester group, oligohistidine group, Strep-tag I, Strep-tag II, desthiobiotin, biotin, chitin, chitin derivatives, chitin binding domain, metal chelate complex, streptavidin, streptactin, avidin and neutravidin.

The first and second functional groups for example can be produced by molecular imprinting. The first and second functional groups can also be active esters, such as the so-called surfmers.

A nanoparticle used according to the invention thus has on its surface a first functional group, which is coupled covalently or non-covalently to a second functional group of a biomolecule that is to be immobilized, the first functional group being a different group than the second functional group. The two groups that are bound together must be complementary to one another, i.e. they must be capable of undergoing covalent or non-covalent binding to one another.

If, according to the invention, an alkyl ketone group, especially methyl ketone or aldehyde group, for example, is used as the first functional group, the second functional group is a hydrazine or hydrazide group. If, conversely, a hydrazine or hydrazide group is used as the first functional group, according to the invention the second functional group is an alkyl ketone, especially a methyl ketone or aldehyde group. If, according to the invention, a thiol group is used as the first functional group, the second complementary functional group is a thioester group. If a thioester group is used as the first functional group, according to the invention the second functional group is a thiol group.

If a metal ion-chelate complex is used as the first functional group according to the invention, the second complementary functional group is an oligohistidine group. If an oligohistidine group is used as the first functional group, the second complementary functional group is a metal ion-chelate complex.

If Strep-tag I, Strep-tag II, biotin or desthiobiotin is used as the first functional group, streptavidin, streptactin, avidin or neutravidin is used as the second complementary functional group. If streptavidin, streptactin, avidin or neutravidin is used as the first functional group, Strep-tag I, Strep-tag II, biotin or desthiobiotin is used as the second complementary functional group.

If in a further embodiment chitin or a chitin derivative is used as the first functional group, a chitin binding domain is used as the second complementary functional group. If a chitin binding domain is used as the first functional group, chitin or a chitin derivative is used as the second complementary functional group.

The aforementioned first and/or second functional groups can, according to the invention, be bound to the biomolecule that is to be immobilized or to the nanoparticle core with the aid of a spacer, or can be introduced onto the nanoparticle core or into the biomolecule by means of a spacer. The spacer thus serves on the one hand for maintaining a distance between the functional group and the core or biomolecule, and on the other hand as a carrier for the functional group. The said spacer can, according to the invention, consist of alkylene groups or ethylene oxide oligomers with 2 to 50 carbon atoms, which in a preferred embodiment has been substituted and possesses heteroatoms.

In a preferred embodiment of the invention, it is envisaged that the second functional groups are a natural component part of the biomolecule, especially of a protein.

In the case of a protein of medium size, i.e. of a size of about 50 kDa with about 500 amino acids, there are about 20 to 30 reactive amino groups, which in principle come into consideration as the functional group for immobilization. In particular this applies to the amino group at the N-terminal end of a protein. All other free amino groups, especially those of the lysine residues, also come into consideration for immobilization. Arginine too, with its guanidium group, comes into consideration as a functional group.

In a further preferred embodiment of the invention it is envisaged to introduce the second functional groups into the biomolecule by methods of genetic engineering, biochemical, enzymatic and/or chemical derivatization or methods of chemical synthesis. Derivatization should be carried out in such a way that the biological activity is preserved after immobilization.

If the biomolecule is a protein, it is possible for example to introduce unnatural amino acids into the protein molecule by methods of genetic engineering or during a chemical protein synthesis, for example together with spacers or linkers. The said unnatural amino acids are compounds that have an amino acid function and a residue R and are not defined by a naturally occurring genetic code, moreover in an especially preferred manner these amino acids have a thiol group. It can also be envisaged according to the invention to modify a naturally occurring amino acid, for example lysine, for example by derivatization of its side chain, especially its primary amino group, with the carboxylic acid function of levulinic acid.

In a further preferred embodiment of the present invention, functional groups can be inserted in a protein by modifying it, with tags, i.e. markers, being attached to the protein, preferably at the C-terminus or the N-terminus. These tags can, however, also be arranged intramolecularly. In particular it is envisaged that a protein is modified so that at least one Strep-tag, for example a Strep-tag I or Strep-tag II or biotin is attached. According to the invention, a Strep-tag also means functional and/or structural equivalents, provided they can bind streptavidin groups and/or its equivalents. The term “streptavidin” thus also encompasses, in the sense of the present invention, its functional and/or structural equivalents. It is also envisaged, according to the invention, to modify a protein by adding on a His-tag, comprising at least three histidine residues, though preferably an oligohistidine group. The His-tag inserted in the protein can then bind to a molecule-specific recognition site that includes a metal-chelate complex.

In a preferred embodiment of the invention it is thus envisaged to effect the binding of proteins, which are modified for example with unnatural amino acids, natural but unnaturally derivatized amino acids or specific Strep-tags, or of antibody-bound proteins to the surfaces of reactive nanoparticles that are complementary thereto, in such a way that a suitable specific, especially non-covalent binding of the proteins to the surfaces and therefore a directed immobilization of the proteins occurs. After alignment of the bioactive molecules via tag binding sites, these molecules can additionally be bound covalently, for example also with a crosslinker such as glutardialdehyde. The protein surfaces become more stable in consequence.

The nanoparticles that are deposited for forming a microstructure on the carrier surface of the functional element have a core, in addition to the surface with the molecule-specific recognition sites. In connection with the present invention, a “core” of a nanoparticle means a chemically inert material that serves as carrier for the molecule that is to be immobilized. According to the invention the core is a compact or hollow particle in the size range from 5 nm to 500 nm.

In a preferred embodiment of the present invention, the core of the nanoparticle used according to the invention consists of an inorganic material such as a metal, for example Au, Ag or Ni, silicon, SiO₂, SiO, a silicate, Al₂O₃, SiO₂.Al₂O₃, Fe₂O₃, Ag₂O, TiO₂, ZrO₂, Zr₂O₃, Ta₂O₅, zeolite glass, indium tin oxide, hydroxylapatite, a Q-dot or a mixture thereof or contains this.

In a further preferred embodiment of the invention the core consists of an organic material or contains this. Preferably the organic polymer is polypropylene, polystyrene, polyacrylate, a polyester of lactic acid or a mixture thereof.

The cores of the nanoparticles used according to the invention can be produced using usual methods known in this special field, such as sol-gel synthesis methods, emulsion polymerization, suspension polymerization etc. Following production of the cores, the surfaces of the cores are provided with the specific first functional groups by chemical modification reactions, for example using usual methods such as graft polymerization, silanization, chemical derivatization etc. One possible way of producing surface-modified nanoparticles in one step consists of using surfmers in emulsion polymerization. Another possibility is molecular imprinting.

Molecular imprinting is to be understood as the polymerization of monomers in the presence of templates, which are able to form a relatively stable complex with the monomer during polymerization. After washing out the templates, the materials thus produced can again specifically bind template molecules, molecular species structurally related to the template molecules or molecules that have groups that are structurally related or identical to the template molecules or parts thereof. A template is therefore a substance that is present in the monomer mixture during polymerization, for which the polymer formed displays an affinity.

Surface-modified nanoparticles are produced in an especially preferred manner according to the invention by emulsion polymerization using surfmers. Surfmers are amphiphilic monomers (surfmer=surfactant+monomer), which can be polymerized on the surface of latex particles, which they stabilize. Reactive surfmers additionally have functionalizable end groups, which can be reacted in mild conditions with nucleophilic substances, such as primary amines (amino acids, peptides, proteins), thiols or alcohols. A large number of biologically active polymeric nanoparticles can be obtained in this way. Publications that reflect the state of the art and the possibilities and limits in the application of surfmers are U.S. Pat. No. 5,177,165, U.S. Pat. No. 5,525,691, U.S. Pat. No. 5,162,475, U.S. Pat. No. 5,827,927 and JP 4 018 929. Works on the synthesis of surfmers with reactive end groups have been published by, among others: Nagai et al. (Polymer 1996, 37(1), 1257-1266; Journal of Colloid and Interface Science 1995, 172, 63-70), Asua et al. (J. Applied Polym. Sci. 1997, 66, 1803-1820) and Guyot et al. (Curr. Opin. Colloid Interface Sci. 1996, 1(5), 580-586).

The density of the first functional groups and the distance between these groups can according to the invention be optimized for each molecule that is to be immobilized. The surroundings of the first functional groups on the surface can also be prepared appropriately for immobilization of a biomolecule that is as specific as possible.

In a preferred embodiment of the invention it is envisaged that additional functions are anchored in the core, and permit simple detection of the nanoparticle cores and therefore of the microstructures, by using suitable methods of detection. These functions can be, for example, fluorescence markers, UV/Vis markers, superparamagnetic functions, ferromagnetic functions and/or radioactive markers. Suitable methods for the detection of nanoparticles include for example fluorescence or UV-Vis spectroscopy, fluorescence or light microscopy, MALDI mass spectroscopy, waveguide spectroscopy, impedance spectroscopy, and electrical and radiometric methods. In a further embodiment it is envisaged that the core surface can be modified by applying additional functions such as fluorescence markers, UV-Vis markers, superparamagnetic functions, ferromagnetic functions and/or radioactive markers. In yet another embodiment of the invention it is envisaged that the core of the nanoparticles is surface-modified with an organic or inorganic layer, which has the first functional groups and the additional functions described above.

In another embodiment of the invention it is envisaged that the core surface has chemical compounds, which serves for steric stabilization and/or prevention of a change of conformation of the molecules that are to be immobilized and/or for preventing the deposition of further biologically active compounds on the core surface. Preferably these chemical compounds are polyethylene glycols, oligoethylene glycols, dextran or a mixture thereof.

According to the invention there is also the possibility of ion-exchange functions being anchored separately or additionally on the surface of the nanoparticle cores. Nanoparticles with ion-exchange functions are suitable in particular for the optimization of MALDI analysis, since disturbing ions can be bound by them.

In a further embodiment of the invention it is envisaged that the biological molecule immobilized on the microstructure of the functional element has markers, which permit simple detection of the biological molecules immobilized on the microstructure using suitable methods of detection. These markers can be, for example, a fluorescence marker, a UV/Vis marker, a superparamagnetic function, a ferromagnetic function and/or a radioactive marker. As noted above, the following can be considered for example as methods of detection for these markers: fluorescence or UV-Vis spectroscopy, MALDI mass spectroscopy, waveguide spectroscopy, impedance spectroscopy, and electrical and radiometric methods.

A further embodiment of the invention relates to a functional element with at least one biological molecule immobilized on the microstructure, with at least one further biological molecule bound covalently or non-covalently to this immobilized molecule. When the molecule immobilized on the microstructure is a protein, it is possible for example for a second protein or an antibody to be bound to it, for example by protein-protein interaction or by antibody-antigen binding. When the molecule immobilized on the microstructure is a nucleic acid, a protein for example can be bound to it.

A further preferred embodiment of the invention relates to a functional element whose microstructure(s) consists/consist of a single nanoparticle layer. A further preferred embodiment of the invention relates to a functional element whose microstructure(s) consists of several superposed layers of the same nanoparticles, each individual layer being bound firmly to the underlying layer via the previously described bonding layers of suitable polymers.

Yet another preferred embodiment of the invention relates to a functional element with several different microstructures arranged on its carrier surface, the said microstructures consisting of nanoparticles with different molecule-specific recognition sites. The said functional elements therefore contain microstructures next to one another, on which different biological molecules are immobilized or can be immobilized. The carrier surface of these functional elements can therefore comprise for example simultaneously microstructures on which proteins are or can be immobilized, and microstructures on which nucleic acids are or can be immobilized. The functional element can, however, also have microstructures on which different proteins or different nucleic acids are or can be immobilized simultaneously.

A further embodiment of the invention relates to a functional element in which the portions of the carrier surface that are not covered by the microstructure have been modified by applying additional functionalities or chemical compounds. These can, in particular, be functionalities or chemical compounds that prevent a nonspecific attachment of biomolecules to the regions of the carrier surface that are not covered by the microstructure. Preferably these chemical compounds are polyethylene glycols, oligoethylene glycols, dextran or a mixture thereof. Especially preferably, the surface of the functional element carrier contains an ethylene oxide layer.

The present invention also relates to a method of production of a functional test according to the invention, in which at least one layer of a bonding agent and then at least one microstructure consisting of nanoparticles with molecule-specific recognition sequences are applied to the surface of a suitable carrier.

It is envisaged according to the invention that the surface of a functional element is prestructured before the layer of bonding agent is applied. After the prestructuring of the carrier surface, a layer of a compound that prevents non-specific attachment of biological molecules to the carrier surface can then be applied on the prestructured carrier surface. This is preferably an ethylene oxide layer.

In a preferred embodiment of the invention it is envisaged that the surface of the carrier of the functional element according to the invention is activated after the prestructuring and before applying the layer of bonding agent. According to the invention, the activation can also include a cleaning operation. Activation of the surface of the carrier of the functional element can be effected according to the invention using a chemical method, in particular using primers or using acids or bases. However, according to the invention there is also the possibility of activating the surface of the carrier using a plasma. Activation can also comprise the application of a self-assembled monolayer.

Basically the following two embodiments can be used for production of the microstructures on the carrier surface of the functional element according to the invention.

In the first embodiment of the method according to the invention for production of a functional element according to the invention it is envisaged that the bonding agent is first applied in a structured manner on the surface of the carrier. “Structured” means, in the context of the invention, that a bonding agent layer of defined shape and area is applied to the carrier surface, and the bonding agent layer thus applied defines the portion of the carrier surface that is to be covered later by the microstructure. The microstructure is then applied by dipping the functional element carrier into a nanoparticle suspension, with the nanoparticles only adhering to the structured applied bonding agent layer, and not to the portions of the carrier surface that do not have a bonding agent layer. In this way a microstructure is produced that is defined with respect to shape and area.

It is envisaged according to the invention that the structured bonding agent layer is applied for example by means of a ring/pin printer, an inkjet method, for example a piezo- or thermo-process, or a microcontact printing process. When using a lithographic process, especially the photolithography or the micropen lithography process, the carrier surface is covered with the bonding agent and then the bonding agent layer thus produced is structured by the lithographic process. With a suitable choice of bonding agent, the microstructure to be applied can be designed so that the microstructure or parts thereof are switchable from outside, for example by altering the pH value, the ion concentration or the temperature, and can be detached again subsequently (debond on command). In this way a microstructure can for example be transferred from one functional element to another.

Stable nanoparticle suspensions can be prepared simply, by suspending the nanoparticles in liquids, especially aqueous media, if necessary using additional constituents, e.g. pH agents, suspension aids etc.

The second embodiment of the method according to the invention for the production of a functional element envisages firstly providing the carrier with a bonding agent layer that covers the entire carrier surface. This can be effected for example by dipping the carrier in a suspension or solution of the bonding agent. Then the microstructure is produced by the structured application of a nanoparticle suspension for example using a ring/pin printer, an inkjet process, for example a piezo- or thermo-process, or a microcontact printing process, so that a microstructure that is defined with respect to shape and area is produced. When a lithographic process is used, especially the photolithography or the micropen lithography process, the carrier surface is covered with the nanoparticle suspension and then the nanoparticle layer thus produced is structured by means of the lithographic process.

The nanoparticles applied to the carrier of a functional element according to the invention for the production of microstructures can be biofunctionalizable nanoparticles, i.e. nanoparticles that merely have molecule-specific recognition sites, but not yet with any biological molecules bound to them. In accordance with the invention it is, however, also possible, for structuring the carrier surface, to use biofunctionalized nanoparticles, i.e. nanoparticles on whose molecule-specific recognition sites biological molecules are already immobilized, retaining their biological activity. It is envisaged according to the invention that the immobilized biological molecule is in particular a protein, a PNA molecule or a nucleic acid.

A further preferred embodiment of the invention envisages applying the same bonding agent and/or the same nanoparticles several times, to produce firmly adhering, multilayer microstructures. According to the invention it is possible to repeat one of the methods described above up to ten times. The methods described above can, however, also be repeated using different bonding agents and/or different nanoparticles, to produce functional elements with different microstructures, which have different functions.

A further preferred embodiment of the invention envisages arranging superparamagnetic or ferromagnetic iron-oxide nanoparticles in a structured manner on a carrier surface using a magnetic field, thereby constructing microstructures, in particular nanoscopic conducting tracks, directly.

After the nanoparticles have been applied to the carrier surface of the functional element there is, according to the invention, the possibility of then converting the particles further. If, for example, the particles contain reactive esters, these can be used for the direct binding of proteins. The nanoparticles can, however, also be converted in order to provide them with additional functions. According to the invention there is also the possibility of additionally fixing the microstructures consisting of nanoparticles, for example by crosslinking the particles covalently with one another and/or with the bonding agent.

The present invention also relates to the use of the functional element according to the invention for investigating an analyte in a sample and/or for its isolation and/or purification therefrom, with the functional element according to the invention being designed for example as a gene array or gene chip or as a protein array. In connection with the present invention, an “analyte” means a substance for which the nature and amount of its individual constituents are to be determined and/or which is to be separated from mixtures. The analyte comprises, in particular, proteins, nucleic acid, carbohydrates and the like. In a preferred embodiment of the invention the analyte is a protein, peptide, active substance, harmful substance, toxin, pesticide, antigen or a nucleic acid. “Sample” means an aqueous or organic solution, emulsion, dispersion or suspension, which contains an analyte as defined above in isolated or purified form or as a constituent of a complex mixture of various substances. A sample can be, for example, a biological fluid, such as blood, lymph, tissue fluid etc., i.e. a fluid that has been taken from a living or dead organism, organ or tissue. A sample can, however, also be a culture medium, for example a fermentation medium, in which organisms, for example microorganisms, or human, animal or plant cells have been cultivated. However, a sample in the sense of the invention can also be an aqueous solution, emulsion, dispersion or suspension of an isolated and purified analyte. A sample can already have undergone purification steps, but it can also be in unpurified form.

The present invention therefore relates to the use of the functional element according to the invention for carrying out methods of analysis and/or detection, these methods being MALDI mass spectroscopy, fluorescence or UV-Vis spectroscopy, fluorescence or light microscopy, waveguide spectroscopy or an electrical method such as impedance spectroscopy.

The present invention also relates to the use of a functional element according to the invention for controlling cellular adhesion or cellular growth.

The present invention also relates to the use of a functional element according to the invention for detecting and/or isolating biological molecules. For example, a functional element according to the invention, with a preferably single-stranded nucleic acid immobilized on its microstructures, can be used for detecting a complementary nucleic acid in a sample and/or for isolating this complementary nucleic acid. A functional element according to the invention, with a protein immobilized on its microstructures, can be used for example for detecting and/or for isolating a protein that interacts with the immobilized protein, from a sample.

The present invention also relates to the use of a functional element according to the invention for the development of pharmaceutical preparations. The invention also relates to the use of the functional elements according to the invention for investigating the effects and/or side-effects of pharmaceutical preparations. The functional elements according to the invention can also be used for diagnosing diseases, for example for identifying pathogens and for identifying mutated genes, which lead to the development of diseases. A further possible use of the functional elements according to the invention is in the investigation of microbiological contamination of surface waters, groundwater and soils. Similarly, the functional elements according to the invention can be used for investigating the microbiological contamination of foodstuffs or animal feed.

A further preferred use of the functional elements according to the invention is the use of the functional element according to the invention as an electronic component, for example as a molecular circuit etc., in medical engineering or in a biocomputer. The use of the functional element according to the invention as an optical storage device in optical information processing, with the functional element according to the invention in particular comprising photoreceptor proteins immobilized on microstructures that can convert light to a signal directly, is especially preferred.

Further advantageous embodiments of the invention follow from the subclaims.

The invention will be explained in more detail on the basis of the following diagrams and examples.

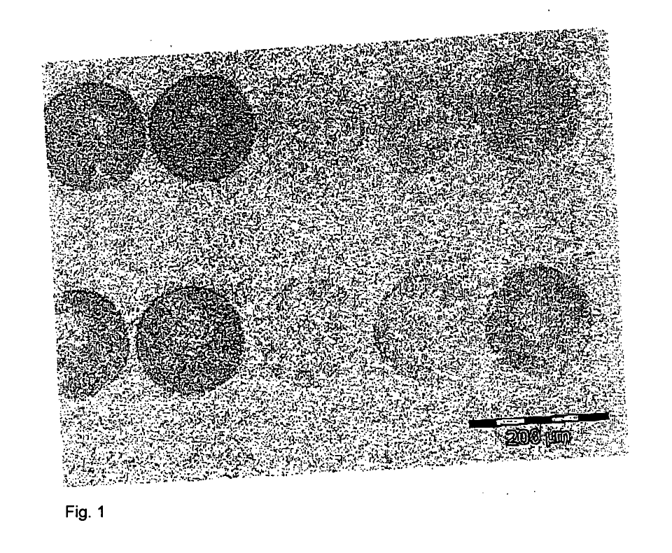

FIG. 1 shows a light-microscope micrograph of microstructured microspots with a diameter from 150 μm to 155 μm of nanoparticles on a silicon carrier.

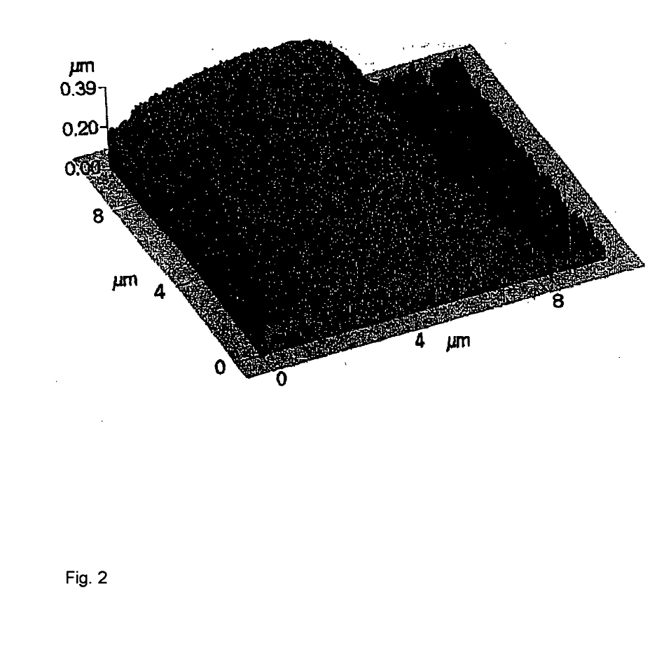

FIG. 2 shows a 3D image, obtained with a scanning force microscope, of the edge of a microspot of a nanoparticle suspension applied 10 times.

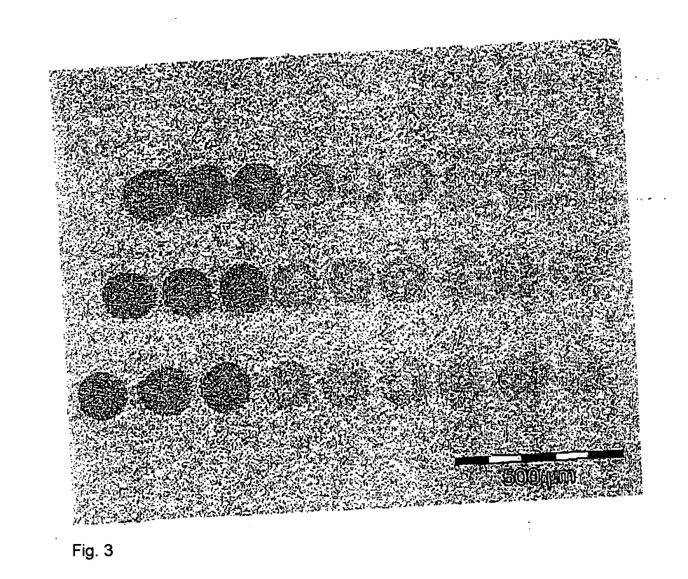

FIG. 3 shows a light-microscope micrograph of microstructured microspots with a diameter from 140 μm to 145 μm of nanoparticles on a glass carrier.

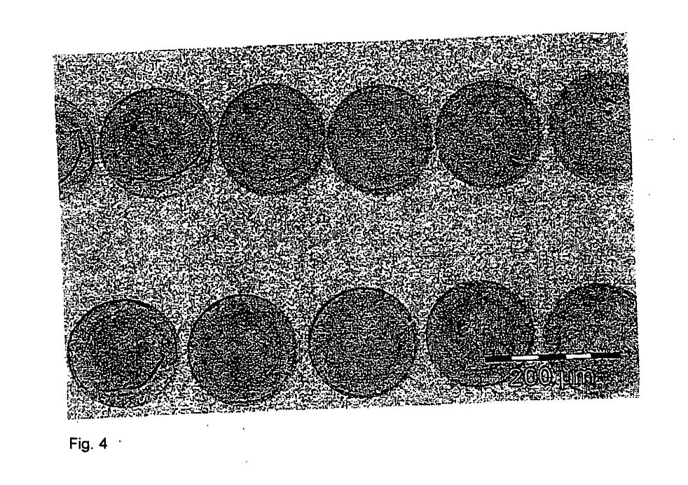

FIG. 4 shows a light-microscope micrograph of microstructured microspots with a diameter from 160 μm to 166 μm of nanoparticles on a glass carrier.

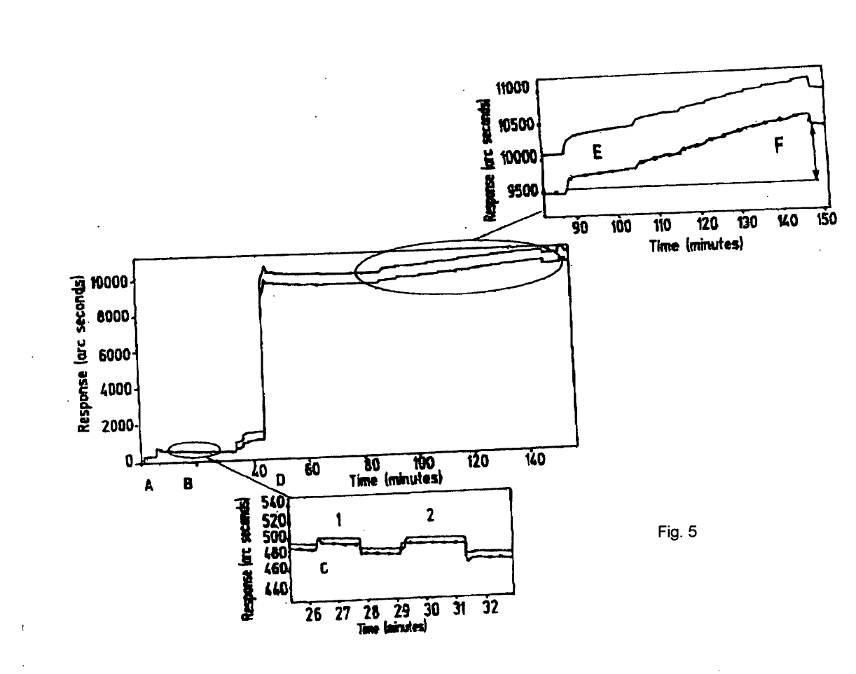

FIG. 5 is a graph (waveguide spectroscope) showing the retained function of immobilized nanoparticles. The base line is in each case 0.1 M phosphate buffer. A) Addition of 0.02 M PDADMAC in 0.1 M NaCl. B) Addition of 0.01 M SPS in 0.1 M NaCl. C) Addition of 0.5 μm (1) and 1 μm (2) streptavidin as control test. No nonspecific binding can be seen. D) Addition of 0.5% (w/v) of the nanoparticles. E) Binding of streptavidin 0.5-3 μm. F) Difference in arcsec. This is the amount that was bound to the particles specifically.

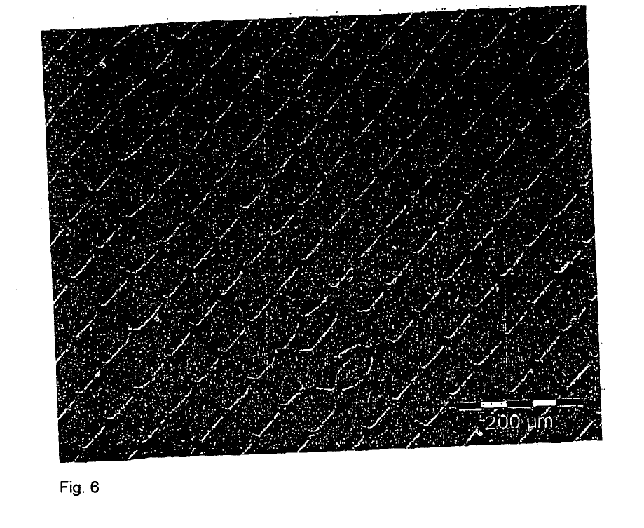

FIG. 6 shows a light-microscope differential interference-contrast micrograph of microstructures. The nanoparticles are localized on the dark areas (lands). The width of the lands is between 10 μm and 13 μm.

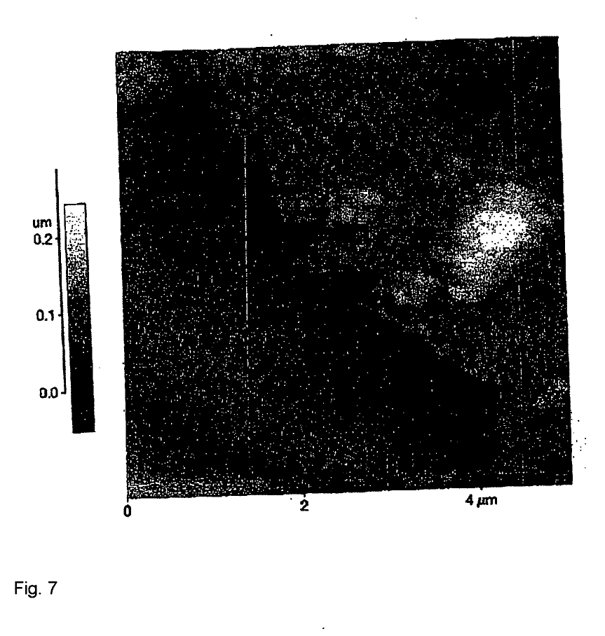

FIG. 7 shows a scanning-force-microscope micrograph (5×5 μm²) of microstructures.

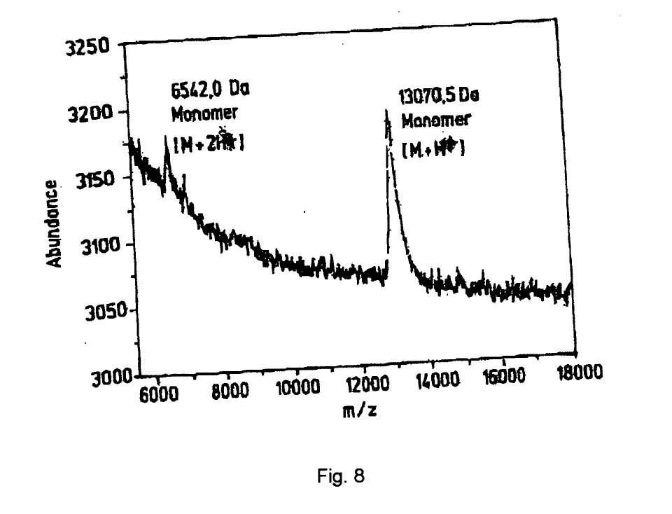

FIG. 8 shows the results of a MALDI-TOF mass spectroscopy analysis using a sample carrier that had monolayers of protein-coated nanoparticles (SPS-PDADMAC-streptavidin-modified particles).

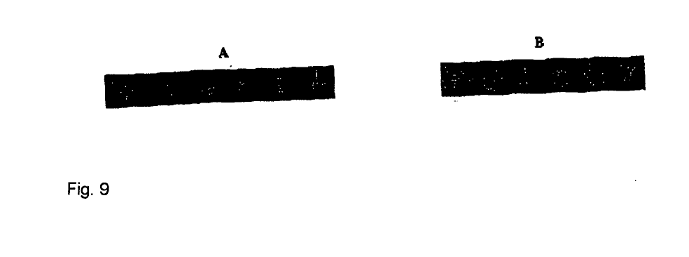

FIG. 9 shows in (A) the hybridization of various oligonucleotides after binding to a commercial chip in each case with a complementary fluorescence-labeled DNA and in (B) the hybridization of the same oligonucleotides after binding to a nanoparticle chip surface according to the invention in each case with a complementary fluorescence-labeled DNA. Comparison of the two chip surfaces shows that the surface modification with nanoparticles according to the invention leads to a definite increase in signal intensity.

### EXAMPLE 1

Synthesis of Silica Particles

12 mmol tetraethoxysilane and 90 mmol NH₃ are added to 200 ml ethanal. This is then stirred at room temperature for 24 h. Then the particles are cleaned by repeated centrifugation.

This gives 650 mg of silica particles with an average particle size of 125 nm.

### EXAMPLE 2

Synthesis of Magnetic Iron-Oxide Particles

20 ml of a 1 M FeCl₃ solution and 5 ml of a 2 M FeSO₄ solution in 2 M HCl are added, stirring vigorously, to 250 ml of a 0.7 M NH₃ solution. Stirring is continued for 30 min and the black solid is washed with 200 ml water. Then the precipitate is stirred with 100 ml 2 M HNO₃ for 30 min and is washed 3 times with 100 ml water. The superparamagnetic iron-oxide nanoparticles are resuspended in 50 ml of a 0.1 M tetramethylammonium hydroxide solution.

This gives 2 g of iron-oxide particles with an average particle size of 10 nm.

### EXAMPLE 3

Synthesis of Magnetic Composite Particles

50 mg of the magnetic iron-oxide nanoparticles obtained above are washed twice with 5 ml ethanol and then taken up in 200 ml ethanol. Then 12 mmol tetraethoxysilane and 90 mmol NH₃ are added. After stirring for 24 h at room temperature, the particles are cleaned by repeated centrifugation.

This gives 600 mg of magnetic composite particles with an average particle size of 150 nm.

### EXAMPLE 4

Synthesis of Fluorescent Particles

190 μmol fluorescein amine and 170 μmol isocyanatopropyltriethoxysilane in 50 ml ethanol are boiled under reflux for 3 h. 3 mmol tetraethoxysilane and 880 μl of the silane dye solution are added to 50 ml ethanol. After addition of 22.5 mmol NH₃ it is stirred at room temperature for 24 h. Then the particles are cleaned by repeated centrifugation.

This gives 160 mg of silica particles with an average particle size of 110 nm.

### EXAMPLE 5

Synthesis of Organic Polymer Nanoparticles

50 mg of the emulsifier p-(11-acrylamido)-undecenoyl-oxyphenyl-dimethylsulfonium-methylsulfate is dissolved in 30 ml water, with stirring. Argon is led through this solution for one hour. Then 1.8 ml methyl methacrylate is added, while stirring. The resulting emulsion is heated to 60° C. Polymerization is started by adding 10 mg of 2,2′-azobis(2-amidinopropane) dihydrochloride. After 5 h the particle suspension is cooled and the particles are cleaned by centrifugation.

We obtain 1.6 g of particles with an average particle size of 145 nm. The particles carry covalently-coupled sulfonium groups on their surface (zeta potential in phosphate buffer pH 7.0: +22 mV) and are capable of binding nucleophiles.

### EXAMPLE 6

Surface Modification of Particles (Amino-Functionalized Surface)

10 vol. % of 25% ammonia is added to a 1 wt. % aqueous suspension of the particles obtained in one of the examples 1 to 4. Then 20 wt. % of aminopropyl triethoxysilane, relative to the particles, is added, then stirred at room temperature for 1 h. The particles are cleaned by repeated centrifugation and carry functional amino groups on their surface (zeta potential in 0.1 M acetate buffer: +35 mV).

### EXAMPLE 7

Surface Modification of Particles (Amino-Functionalized Organis Polymer Particles)

10 mg og the particles obtained in Example 5 are taken up in 50 μl of a 10 mmol phosphate buffer (ph 7.8) and 950 μl of a 1 M ethylenediamine solution in 10 mmol phosphate buffer (ph 7.8) is added. It is then shaken for 2 h at room temperature. This is followed by cleaning by centrifugation. The particles carry covalently bound amino groups on their surface.

### EXAMPLE 8

Surface Modification of Particles (Carboxy-Functionalized Surface)

10 ml of a 2 wt. % suspension of amino-functionalized nanoparticles is taken up in tetrahydrofuran. 260 mg of succinic anhydride is added. After sonication for 5 min, it is stirred at room temperature for 1 h. Then the particles are cleaned by repeated centrifugation. The resulting silica particles carry functional carboxy groups (zeta potential in 0.1 M acetate buffer of −35 mV) on their surface and have an average particle size of 170 nm.

### EXAMPLE 9

Surface Modification of Particles (Carboxy-Dextran-Modified Particles)

10 mg of amino-functionalized nanoparticles and 1 mg of carboxydextran (Sigma, >55 cps) are placed in 1 ml of 0.1 M morpholino-ethane sulfonic acid buffer (MES, pH: 5.0). 30 μl of N-ethyl-N′-(3-dimethylaminopropyl)-carbodiimide (EDC) solution at a concentration of 500 μmol/ml is added. Then it is shaken for 30 min at room temperature. The particles are washed alternately with MES and TBE buffer (89 mM Tris-(hydroxymethyl)aminomethane, 89 mM boric acid, 2 mM ethylenediamine tetraacetic acid, pH: 8.3) and taken up in 1 ml MES buffer. The resulting silica particles carry functional carboxy groups (zeta potential in 0.1 M acetate buffer of −25 mV) on their surface and have an average particle size of 160 nm.

This dextran surface is especially suitable for the immobilization of proteins, whose tertiary structure is disturbed by adsorption processes on the particle surface.

### EXAMPLE 10

Surface Modification of Particles (Amino-Functionalization of Carboxy-(Dextran)-Particles)

A 1 M ethylenediamine solution in 0.1 M MES buffer (pH 5.0) is prepared. 500 μg of the carboxy-(dextran)-modified particles and 30 μl of a 500 μmol/ml EDC solution in MES buffer are added. Then it is shaken for 3 h at room temperature. Next it is washed several times with MES buffer. This gives particles with an average particle size of 160 nm and a zeta potential of +25 mV in 0.1 M acetate buffer.

The spacer length and the density of the functional groups can be varied by using other amines similarly, for example 4,7,10-trioxa-1,13-tridecanediamine (or higher homologs) or Tris-(2-aminoethyl)-amine.

### EXAMPLE 11

Surface Modification of Particles (Nitrilotriacetic Acid (NTA)-Surface)

10 mg of carboxy-modified particles are washed twice with 1 ml acetonitrile (MeCN) and taken up in 1 ml MeCN. 10 μmol dicyclohexyl carbodiimide and 10 μmol N-hydroxysuccinimide are added. Then it is shaken for 2 h at room temperature. This is followed by washing once with 1 ml cyclohexane and once with 1 ml MeCN. The reaction mixture is taken up in 1 ml MeCN. 4 μmol of N,N-bis-carboxymethyl-L-lysine is added. After shaking for 3 hours at room temperature it is washed once with 1 ml acetonitrile and twice with 1 ml 10 mM phosphate buffer (pH 7.0).

As a result of this processing, on the one hand the density of the functional carboxy groups is increased, and on the other hand Ni²⁺ ions can be bound to this surface by complexing. The surface is then capable of binding His-Tag modified proteins.

### EXAMPLE 12

Surface Modification of Particles (Thiol Surface)

10 mg of carboxy-modified particles are washed twice with 1 ml acetonitrile (MeCN) and are taken up in 1 ml MeCN. 10 μmol of dicyclohexyl carbodiimide and 10 μmol of N-hydroxysuccinimide are added, followed by shaking for 2 h at room temperature. It is washed once with 1 ml cyclohexane and once with 1 ml MeCN and is taken up in 1 ml MeCN. 500 μg cysteine is added, followed by shaking for 3 h at room temperature. It is washed once with 1 ml acetonitrile and twice with 1 ml 10 mM phosphate buffer (pH 7.0).

This surface is suitable for the immobilization of proteins via disulphide bridges.

### EXAMPLE 13

Functionalization For Protein Immobilization (Maleimide-Activated Surface)

500 μg of amino-functionalized particles are resuspended in 1 ml of 10 mM phosphate buffer (pH 7.0). 1.25 μmol sulfosuccinimidyl-4-(N-maleimidomethyl) cyclohexane-1-carboxylate is added. After shaking for 1 h at room temperature, it is washed once with cold 10 mM phosphate buffer (pH 7.0) and the mixture is taken up in 1 ml pf 0.1 M phosphate buffer (pH 7.0).

### EXAMPLE 14

Functionalization For Protein Immobilization (Iodoacetyl-Activated Surface)

500 μg of amino-functionalized particles are resuspended in 1 ml of 10 mM phosphate buffer (pH 7.0). 1.25 μmol of succinimidyl-(4-iodoacetyl)aminobenzoate is added, it is shaken for 1 h at room temperature, washed once with cold 0.1 M phosphate buffer (pH 7.0) and taken up in 1 ml 10 mM phosphate buffer (7.0).

These surfaces are suitable for coupling proteins that carry free thiol groups.

### EXAMPLE 15

Functionaliztion For Protein Immobilization (Biotinylated Surface)

500 μg pf amino-functionalized particles are resuspended in 1 ml of 10 mM phosphate buffer (pH 7.0). 1.25 μmol of succinimidobiotin is added, it is shaken for 1 h at room temperature, washed once with 1 M phosphate buffer (pH 7.0) and taken up in 1 ml of 10 mM phosphate buffer (pH 7.0).

Coating with functional groups is described in examples 13 to 15 so that it takes place quantitatively. Usually, however, these coating operations can be controlled by appropriate choice of the reaction conditions (generally by means of the concentration of the modifier) so that they only take place partially. With an appropriate choice of modifiers it is also possible to have various functional groups next to one another on the particle surface. Examples of this are:
—NH₂ along with —COOH:

In Example 8 the concentration of succinic anhydride is lowered. With appropriate choice of the NH₂/COOH ratio it is possible to vary the isoelectric point of the particle system over a wide range (between 8 and 3). During reaction with proteins, this can be a decisive parameter for controlling the reactivity (systems with like charges repel one another).
—SH along with NTA:

The reaction is carried out as described in examples 11 or 12, but using a mixture of the two modifiers. In this way, proteins bearing His-Tag can be oriented non-covalently in a first step and then this state can be fixed permanently by forming a covalent disulphide bridge.

### EXAMPLE 16

Immobilization of Proteins (Streptavidin-Modified Particles)

2.68 nmol streptavidin is placed in 10 ml of 0.1 m MES buffer (pH 5.0). 5 mg of the particles obtained in Example 8 are added. 2 μmol EDC is added. After shaking for 3 h at room temperature, the particles are washed once with 10 ml MES buffer and once with 10 ml phosphate buffer (pH 7.0). Then the particles are taken up in 10 ml phosphate buffer (pH 7.0).

### EXAMPLE 17

Immobilization of Proteins (Protein G-Modified Particles)

300 μg of protein G is placed in 10 ml of 0.1 m phosphate buffer (pH 7.5). 5 mg of the particles from Example 5 are added, shaking for 3 h at room temperature. Then the particles are washed twice with 10 ml phosphate buffer (pH 7.0) and are taken up in 10 ml phosphate buffer (pH 7.0).

Example 16 and 17 show examples of the immobilization of two proteins by two different routes. Many different proteins can be immobilized by these two routes. Many additional strategies are conceivable with the particles described above, for example activation of carboxy-modified particles with succinimides, and coupling of free cysteines on thiol/maleinimide surfaces. Parallel coupling of different proteins is also possible on multifunctional particles.

All the particles described in examples 1-17 can be used directly for microstructuring.

### EXAMPLE 18

Production of a Gene Array or Protein Array Using a Ring/Pin Plotter on a Prepared Silicon Surface by Means of Nanoparticles with a Silica Core

Using a ring/pin micro-arrayer, the nanoparticle suspension from Example 1 was applied to a silicon carrier. The silicon carriers, cut to size, are stored for 90 min in a 2 vol. % aqueous HELLMANEX® solution at 40° C. This is followed by ultrasonic-bath treatment for 5 min at room temperature and rinsing with deionized water. After drying with nitrogen, the layer thickness is determined using a null ellipsometer.

The samples are then hydroxylated for 30 min in a 3:1 (v/v) NH₃/H₂O₂ solution at 70° C. (NH₃: puriss. p. a., ˜25% in water; H₂O₂: analytical grade, ISO reag., stabilized, deionized water 18 MΩ). Prior to storage in water (max. 3 h) the samples are rinsed thoroughly with deionized water. The substrates are transferred at room temperature to a polyelectrolyte solution (0.02 M polydiallyldimethylammonium chloride (PDADMAC), 0.1 M NaCl, MW=100-200 kDa in deionized water). After 20 min, thorough rinsing with deionized water is carried out. The samples are treated in the ultrasonic bath for 5 min at room temperature, rinsed and dried with nitrogen. The silicon carriers thus prepared are glued onto glass object carriers (approx. 76×26 mm). 1 ml portions of the 1 wt. % aqueous nanoparticle suspensions are in each case shaken for 15 s and dispersed in the ultrasonic bath at room temperature.

The wells of the plates (96 wells of 300 μl, U-shaped) are filled with 100 μl of dispersion. The solutions in the plates are released and after 15 min are transferred with the ring/pin micro-arrayer (GMS 417, Affymetrix, USA) onto the silicon carriers in laboratory conditions. With suitable programming of the ring/pin micro-arrayer, we obtain regularly arranged microspots with a diameter of about 150 μm to 200 μm that are sharply separated from one another, the distance between the individual microstructures being a few micrometers. Analysis of the microspots in the light microscope and/or the scanning-force microscope showed, surprisingly, that when the nanoparticle suspension was applied 10 times in exactly the same position (10 hits per dot) there was particular accumulation of the nanoparticles at the edge of the microspots. A uniform distribution of the nanoparticles within a microspot is obtained in the case of application with 1-2 hits per dot.

FIG. 1 shows a light-microscope micrograph of microstructured microspots with the nanoparticles described above with a diameter of 150 μm to 155 μm and a variable spacing of 5 μm to 20 82 m along the horizontal axis and a spacing of 140 μm along the vertical axis on a silicon carrier.

FIG. 2 shows a 3D micrograph, obtained with the scanning force microscope, of the edge of a microspot of a nanoparticle suspension applied 10 times in exactly the same position, and it can be clearly seen that there is accumulation of the nanoparticles in the edge region of the microspot.

### EXAMPLE 19

Production of a Gene Array or Protein Array Using a Ring/Pin Plotter on a Prepared Glass Surface by Means of Nanoparticles with a Silica Core

Using a ring/pin micro-arrayer, the nanoparticle suspension from Example 1 was applied to a glass carrier. The glass carriers are pretreated as in Example 18 and the 1 wt. % aqueous nanoparticle suspension is also applied to the glass surface as in Example 18.

FIG. 3 shows a light-microscope micrograph of microstructured microspots with the nanoparticles described above with a diameter of 140 μm to 145 μm and a variable spacing of 5 μm to 20 μm along the horizontal axis and a spacing of 140 μm along the vertical axis on a glass carrier.

### EXAMPLE 20

Production of a Gene Array or Protein Array Using a Ring/Pin Printer on a Prepared Glass Surface by Means of Nanoparticles with a PMMA Core

Using a ring/pin micro-arrayer, the nanoparticle suspension from Example 5 was applied to a glass carrier. The glass carriers are stored for 90 min in a 2 vol. % aqueous HELLMANEX® solution at 40° C. This is followed by ultrasonic-bath treatment for 5 min at room temperature and rinsing with deionized water. After drying with nitrogen, the layer thickness is determined using a null ellipsometer. The samples are then hydroxylated for 30 min in a 3:1 (v/v) NH₃/H₂O₂ solution at 70° C. (NH₃: puriss. p. a., ˜25% in water; H₂O₂: analytical grade, ISO reag., stabilized, deionized water 18 MΩ). Prior to storage in water (max. 3 h) the samples are rinsed thoroughly with deionized water. The substrates are transferred at room temperature to polyelectrolyte solution 1 (0.02 M polydiallyldimethylammonium chloride (PDADMAC), 0.1 M NaCl, MW=100-200 kDa in deionized water). After 20 min, thorough rinsing with deionized water is carried out. The glass carriers are then transferred to polyelectrolyte solution 2 (0.01 M polystyrene-sulfonic acid sodium salt (SPS), 0.1 M NaCl, MW=70 kDa in deionized water) for 20 min at room temperature. Storage in polyelectrolyte solution 2 is followed by sonication in deionized water for 5 min at room temperature and drying with nitrogen. The 1 wt. % aqueous nanoparticle suspensions are then applied to the glass surface as in examples 1 and 2.

FIG. 4 shows a light-microscope micrograph of microstructured microspots with the nanoparticles described above with a diameter of 160 μm to 166 μm and a variable spacing of 5 μm to 20 μm along the horizontal axis and a spacing of 140 μm along the vertical axis on a glass carrier.

FIG. 5 is a graph (waveguide spectroscope) showing the retained function of the immobilized nanoparticles obtained in Example 5. The base line is in each case 0.1 M phosphate buffer. A) Addition of 0.02 M PDADMAC in 0.1 M NaCl. B) Addition of 0.01 M SPS in 0.1 M NaCl. C) Addition of 0.5 μm (1) and 1 μm (2) streptavidin as control test. No nonspecific binding can be seen. D) Addition of 0.5% (w/v) of the nanoparticles. E) Binding of streptavidin 0.5-3 μm. F) Difference in arcsec. This is the amount that could be bound to the particles specifically.

### EXAMPLE 21

Production of a Gene Array or Protein Array Using a Lithographic Process

Silicon carriers, that have been coated as described in Example 18, are placed under a mercury UV lamp (Pen-Ray, UVP, USA) at a distance of 2 cm. The bridge of the lamp is inclined at 45° to the vertical. After fitting the copper grid (Plano, Germany, d=3.05 mm) the PDADMAC-coated carriers are irradiated for 45 min. 1 ml of the 1 wt. % aqueous nanoparticle suspension obtained in Example 6 is in each case shaken for 15 s and dispersed in the ultrasonic bath at room temperature. The copper grids are tapped away from the carrier and in each case 40 μl of the suspension is applied to the structured area. After 10 min in laboratory conditions, the samples are transferred to deionized water. This is followed by drying for 1 min in a nitrogen stream.

FIG. 6 shows a light-microscope differential-interference-contrast micrograph of the microstructures thus obtained. The nanoparticles are localized on the dark regions (lands). The width of the lands is between 10 μm and 13 μm.

FIG. 7 shows a scanning-force-microscope micrograph (5×5 μm²) of the microstructures obtained. The sharp demarcation between the land with nanoparticles and the uncoated substrate is clearly visible.

### EXAMPLE 22

MALDI-TOP Mass Spectroscopy Analysis Using a Sample Carrier with Monolayers of Protein-Coated Nanoparticles

This example shows that sample carriers that have monolayers of protein-coated nanoparticles can be used in MALDI-TOF mass spectroscopy directly. The analysis is not disturbed by the bonding layer nor by the nanoparticles.

Sample Preparation

The gold surface of the MALDI sample carrier was pretreated first in acetone, then in a 1:1 mixture of isopropanol (HPLC grade) and 0.02 N HCl and then in isopropanol (HPLC grade) (in each case 10 min in the ultrasonic bath). This is followed by drying with compressed air. The cleaned sample carrier was then dipped in SPS solution (0.01 M SPS, 0.1 M NaCl) for 20 min, washed with deionized water and dried. The sample carrier was then dipped in a suspension of the streptavidin-modified particles obtained in Example 16 (0.5 mg/ml) for 40 min. At this pH value the charge on the particles is −20 mV. The carrier was washed with deionized water and dried. Then 0.5 pl of saturated matrix (3,5-dimethoxy-4-hydroxy-cinnamic acid, dissolved in a 6:4 (v/v) mixture of 0.1% trifluoroacetic acid (TFA, Fluka, p.a.) and acetonitrile (Baker, HPLC grade) were applied, air-dried and then measured in an LD-TOF-MS (HP G 2025A LD-TOF modified with a time-lag-focusing (TLF) unit (Future, supplier: GSG); data acquisition with Le Croy-500 MHz oscilloscope; external calibration; error 0.1%).

Results

The results of the MALDI-TOF-MS analysis are shown in FIG. 8. Two peaks for streptavidin can be seen. The peaks at 13070.5 Da and 6542 Da can be ascribed to the singly charged and doubly charged streptavidin monomer. Accordingly, a molecular weight of 52280 Da is found for the tetrameric streptavidin. The PDADMAC and SPS polyelectrolytes do not disturb detection of the protein.

### EXAMPLE 23

Production of a DNA Chip for Hybridizations and Solid-Phase Enzyme Reactions for the Detection of Point Mutations and SNPs and for Transcription Investigations

The particles obtained in Example 6 were applied, as described in Example 18, to a planar chip surface using the layer-by-layer method. Then using a micro-arrayer, i.e. a device for the production of micro-arrays (pin-and-ring or capillary pins), DNA probes were applied to the nanoparticle carriers thus prepared. Application was effected using DNA solutions in 10 mM Tris/Cl, pH 8.0, 40% DMSO (Vol./Vol.), containing 50 μm DNA in each case. Fixing and post-treatment were carried out as described in Diehl, Grahlmann, Beier and Hoheisel, Nucleic Acids Res., 29 (2001), 7, e38. The DNA chips thus prepared were then used for hybridizations with fluorescence-labeled, reverse-complementary DNA fragments and for enzyme reactions on the chip according to the APEX principle (arrayed primer extension; cf. Pastinen et al., Genome Res., 10 (2000), 1031-1042). As a control, the same DNA probes were applied to poly-L-lysine-coated glass slides in accordance with standard protocols. The control chips thus produced were then also used for hybridizations with fluorescence-labeled, reverse-complementary DNA fragments and for enzyme reactions.

FIG. 9 shows the results of hybridization using the control DNA chip in comparison with the nanoparticle DNA chip according to the application. As can be seen from FIG. 9, the signal intensity in hybridization is significantly increased by the particulate surface according to the invention.

1. A functional element, comprising a carrier with a surface and at least one microstructure on the carrier surface, where the microstructure consists of individual components in the form of nanoparticles, which have molecule-specific detection sites with one or more first functional groups, to which biologically functioning or active molecules with complementary second functional groups which bind to the first functional groups can be bound in a directional manner and thus make possible the addressability of the microstructure, and where, between the surface of the carrier and the microstructure, at least one layer of a bonding agent, selected from a polymer or a plasma layer with charged or uncharged chemically reactive groups, is provided to ensure permanent adherence of the nanoparticles.

2. The functional element as claimed in claim 1, wherein the microstructure covers a portion of the carrier surface and at least one of the area/length parameters of the covered portion of the carrier surface is smaller than 999 μm and at least 10 nm.

3. The functional element as claimed in claim 1, wherein the carrier and/or the surface of the carrier consists of a metal, metal oxide, polymer, semiconductor material, glass and/or ceramic.

4. The functional element as claimed in claim 1, wherein the surface of the carrier is planar.

5. The functional element as claimed in claim 1, wherein the surface of the carrier is pre-structured.

6. The functional element as claimed in claim 1, wherein the surface of the carrier has a layer of a chemical compound that prevents nonspecific attachment of biological molecules to the carrier surface.

7. The functional element as claimed in claim 1, wherein a layer of a bonding agent is arranged between the carrier surface and the microstructure.

8. The functional element as claimed in claim 7, wherein the bonding agent is a polymer with charged or uncharged chemically reactive groups.

9. The functional element as claimed in claim 8, wherein the polymer is a hydrogel.

10. The functional element as claimed in claim 7, wherein the bonding agent is a plasma layer with charged or uncharged chemically reactive groups.

11. The functional element as claimed in claim 7, wherein the bonding agent is a self-assembled monolayer based on silane or thiol.

12. The functional element as claimed in claim 7, wherein the bonding agent is switchable by altering the pH value, the ion concentration or the temperature.

13. The functional element as claimed in claim 1, wherein the nanoparticles comprise a core and a surface that has the molecule-specific recognition sites.

14. The functional element as claimed in claim 13, wherein one or more biologically active molecules are bound to the molecule-specific recognition sites.

15. The functional element as claimed in claim 14, wherein the biologically active molecules are bound covalently and/or non-covalently.

16. The functional element as claimed in claim 14, wherein the molecules are bound preserving their biological activity.

17. The functional element as claimed in claim 14, wherein the bound molecules are proteins, nucleic acids, PNA molecules or fragments thereof.

18. The functional element as claimed in claim 16, wherein the proteins are antibodies, antigens, enzymes, cytokines or receptors.

19. The functional element as claimed in claim 13, wherein the molecule-specific recognition sites comprise one or more first functional groups and the bound molecules comprise complementary second functional groups that bind the first functional groups.

20. The functional element as claimed in claim 19, wherein the first functional groups and the complementary second functional groups that bind the first functional groups are selected from the group comprising active ester, alkyl ketone group, aldehyde group, amino group, carboxy group, epoxy group, maleinimide group, hydrazine group, hydrazide group, thiol group, thioester group, oligohistidine group, Strep-tag I, Strep-tag II, desthiobiotin, biotin, chitin, chitin derivatives, chitin binding domain, metal chelate complex, streptavidin, streptactin, avidin and neutravidin.

21. The functional element as claimed in claim 19, wherein the first and the second functional groups are produced by molecular imprinting.

22. The functional element as claimed in claim 19, wherein the first functional groups are a component part of a spacer or are bound via spacers to the surface of the nanoparticles.

23. The functional element as claimed in claim 19, wherein the complementary second functional groups are a component part of a spacer or are bound via spacers to the molecules.

24. The functional element as claimed in claim 13, wherein the core of the nanoparticles consists of or contains an organic material.

25. The functional element as claimed in claim 24, wherein the organic material is an organic polymer.

26. The functional element as claimed in claim 24, wherein the organic polymer is polypropylene, polystyrene, polyacrylate or a mixture thereof.

27. The functional element as claimed in claim 13, wherein the core consists of or contains an inorganic material.

28. The functional element as claimed in claim 27, wherein the inorganic material is a metal such as Au, Ag or Ni, silicon, SiO₂, SiO, a silicate, A1₂O₃, SiO₂.Al₂O₃, Fe₂O₃, Ag₂O, TiO₂, ZrO₂, Zr₂O₃, Ta₂O₅, zeolite hydroxylapatite, a Q-Dot or a mixture thereof.

29. The functional element as claimed in claim 13, wherein the core has a size from 5 nm to 500 nm.

30. The functional element as claimed in claim 13, wherein the core has at least one additional function.

31. The functional element as claimed in claim 30, wherein the additional function is anchored in the core and is a fluorescence marker, a UV/Vis marker, a superparamagnetic function, a ferromagnetic function and/or a radioactive marker.

32. The functional element as claimed in claim 30, wherein the surface of the core is modified with an organic or inorganic layer containing the first functional groups, which has a fluorescence marker, a UV/Vis marker, a superparamagnetic function, a ferromagnetic function and/or a radioactive marker.

33. The functional element as claimed in claim 30, wherein the surface of the core has a chemical compound, which serves for steric stabilization and/or for preventing a change of conformation of the immobilized molecules and/or for preventing the attachment of a further biologically active compound to the core.

34. The functional element as claimed in claim 33, wherein the chemical compound is a polyethylene glycol, an oligoethylene glycol, dextran or a mixture thereof.

35. The functional element as claimed claim 14, wherein the bound molecules have a marker.

36. The functional element as claimed in claim 15, wherein further molecules are bound to the bound molecules.

37. The functional element as claimed in claim 1, wherein the microstructure consists of a nanoparticle layer.

38. The functional element as claimed in claim 1, wherein the microstructure consists of several nanoparticle layers.

39. The functional element as claimed in claim 1, wherein several microstructures, which consist of nanoparticles with different molecule-specific recognition sites, are arranged on the carrier surface.

40. The functional element as claimed in claim 39, wherein various molecules are bound to the microstructures.

41. The functional element as claimed in claim 1, obtainable by applying one or more microstructures to the carrier surface using a ring/pin printer.

42. The functional element as claimed in claim 1, obtainable by applying one or more microstructures to the carrier surface using a lithographic process.

43. The functional element as claimed in claim 42, wherein the lithographic process is photolithography.

44. The functional element as claimed in claim 42, wherein the lithographic process is micropen lithography.

45. The functional element as claimed in claim 1, obtainable by applying one or more microstructures to the carrier surface using an inkjet process.

46. The functional element as claimed in claim 1, obtainable by applying one or more microstructures using a microcontact printing process.

47. A method for the production of a functional element as claimed in claim 1, wherein at least one layer of a bonding agent and then at least one microstructure consisting of nanoparticles with molecule-specific recognition sites are applied to the surface of a carrier.

48. The method as claimed in claim 47, wherein the surface of the carrier is cleaned and/or activated before applying the layer of bonding agent.

49. The method as claimed in claim 48, wherein the carrier surface is activated chemically.

50. The method as claimed in claim 49, wherein the carrier surface is provided with charges.

51. The method as claimed in claim 49, wherein the carrier surface is activated after applying a primer.

52. The method as claimed in claim 49, wherein a self-assembly layer is applied to the carrier surface.

53. The method as claimed in claim 48, wherein the carrier surface is activated by means of a plasma.

54. The method as claimed in claim 47, wherein a layer of bonding agent defined with respect to shape and area is applied to the carrier surface and the carrier is then dipped into a nanoparticle suspension, so that a microstructure that is defined with respect to shape and area is produced through adherence of the nanoparticles to the applied layer of bonding agent.

55. The method as claimed in claim 54, wherein the layer of bonding agent defined with respect to shape and area is applied by means of a ring/pin printer, a lithographic process, an inkjet process or a microcontact printing process.

56. The method as claimed in claim 47, wherein the carrier is dipped into a suspension or solution of the bonding agent, so that a layer of bonding agent covering the whole carrier surface is produced, and then the nanoparticles are applied in such a way that a microstructure defined with respect to shape and area is produced.

57. The method as claimed in claim 56, wherein the microstructure defined with respect to shape and area is applied by means of a ring/pin printer, a lithographic process, an inkjet process or a microcontact printing process.

58. The method as claimed in claim 47, wherein the bonding agent and the nanoparticles are applied to the carrier surface several times.

59. The method as claimed in claim 47, wherein biologically active molecules are bound to the molecule-specific recognition sites of the nanoparticles before the nanoparticles are applied.

60. The method as claimed in claim 47, wherein biologically active molecules are bound to the molecule-specific recognition sites of the nanoparticles after application of the nanoparticles.

61. The method as claimed in claim 47, wherein biologically active molecules are bound to the molecule-specific recognition sites of the nanoparticles before and after application of the nanoparticles.

62. The method as claimed in claim 59, wherein the binding of the biologically active molecules to the molecule-specific recognition sites of the nanoparticles is effected by bringing the molecule-specific recognition sites of the nanoparticles, which have first functional groups, into contact with the molecules that have complementary second functional groups that bind the first functional groups, in such a way that covalent and/or non-covalent bonds are effected between the functional groups of the molecule-specific recognition sites and the molecules.

63. The method as claimed in claim 62, wherein the first functional groups and the complementary second functional groups that bind the first functional groups are selected from the group comprising active ester, alkyl ketone group, aldehyde group, amino group, carboxy group, epoxy group, maleinimide group, hydrazine group, hydrazide group, thiol group, thioester group, oligohistidine group, Strep-tag I, Strep-tag II, desthiobiotin, biotin, chitin, chitin derivatives, chitin binding domain, metal chelate complex, streptavidin, streptactin, avidin and neutravidin.

64. The method as claimed in claim 59, wherein the biologically active molecules are bound while retaining their biological activity.

65. The method as claimed in claim 59, wherein the molecules are proteins, antigens, nucleic acids, PNA molecules or fragments thereof.

66. (Canceled)

67. (Canceled)

68. (Canceled)

69. (Canceled)

70. (Canceled)

71. (Canceled)

72. (Canceled)

73. (Canceled)

74. (Canceled)

75. (Canceled

76. (Canceled)

77. (Canceled)

78. A method of detection comprising the step of using a functional element as claimed in claim 1 or a functional element produced by the method of claim 47.

79. (Canceled)

80. A method of controlling cellular adhesion or cellular growth comprising the step of using a functional element as claimed in claim 1 or a functional element produced by the method of claim 47.

81. (Canceled)

82. A method of developing pharmaceutical preparations comprising the step of using a functional element as claimed in claim 1 or a functional element produced by the method of claim 47.

83. (Canceled)

84. A method for analyzing the effects or side effects of pharmaceutical preparations comprising the step of using a functional element as claimed in claim 1 or a functional element produced by the method of claim 47.

85. (Canceled)

86. A method for diagnosing disease comprising the step of using the functional element of claim 1 or a functional element produced by the method of claim 47.

87. (Canceled)

88. A method of analyzing microbiological contamination of samples comprising the step of using the functional element of claim 1 or a functional element produced by the method of claim 47.

89. (Canceled)

90. A biocomputer including, as an electronic component thereof, the functional element of claim 1 or a functional element produced by the method of claim 47.

91. (Canceled)

92. The method as claimed in claim 78, wherein the detection method is MALDI mass spectroscopy, fluorescence or UV-Vis spectroscopy, fluorescence or light microscopy, waveguide spectroscopy, impedance spectroscopy or another electrical method.

93. The method as claimed in claim 86, wherein pathogens are identified.

94. The method as claimed in claim 86, wherein mutated genes in a human being or an animal are identified.

95. The method as claimed in claim 88, wherein the sample is a water sample or a soil sample.

96. The method as claimed in claim 88, wherein the sample is obtained from foodstuff or animal feed.
